(19) United States
(12) Patent Application Publication
(10) Pub. No.: US 2010/0196283 A1
(43) Pub. Date: Aug. 5, 2010

(54) METHOD AND IMAGING MEDIUM FOR USE IN THE METHOD

(76) Inventors: Matilde H. Lerche, Fredriksberg (DK); Anna Gisselsson, Lund (SE); Georg Hansson, Vellinge (SE); Sven Mansson, Bjarred (SE); Rene in't Zandt, Sodra Sandby (SE); Magnus Karlsson, Malmo (SE); Pernille R. Jensen, Kobenhavn (DK)

Correspondence Address: GE HEALTHCARE, INC., IP DEPARTMENT 101 CARNEGIE CENTER, PRINCETON, NJ, 08540-6231, US

(21) Appl. No.: 12/670,660

(22) PCT Filed: Jul. 25, 2008
(86) PCT No.: PCT/EP08/59763

§ 371 Date: Jan. 26, 2010

(30) Foreign Application Priority Data
Jul. 26, 2007 (NO) 20073920
Sep. 25, 2007 (NO) 20074887

(51) Int. Cl. A61K 49/20; A61K 49/10; C07C 69/66; A61P 43/00; C12Q 1/02; G01R 33/48

(52) U.S. Cl. 424/9.36; 560/179; 435/29; 324/309

(57) ABSTRACT

The invention relates to a method of ¹³C-MR detection using an imaging medium comprising hyperpolarised ¹³C-lactate and to an imaging medium containing hyperpolarised ¹³C₁-lactate for use in said method.

The invention relates to a method of ¹³C-MR detection using an imaging medium comprising hyperpolarised ¹³C-lactate and to an imaging medium containing hyperpolarised ¹³C₁-lactate for use in said method.

Magnetic resonance (MR) imaging (MRI) is a technique that has become particularly attractive to physicians as images of a patients body or parts thereof can be obtained in a non-invasive way and without exposing the patient and the medical personnel to potentially harmful radiation such as X-rays. Because of its high quality images and good spatial and temporal resolution, MRI is a favourable imaging technique for imaging soft tissue and organs.

MRI may be carried out with or without MR contrast agents. However, contrast-enhanced MRI usually enables the detection of much smaller tissue changes which makes it a powerful tool for the detection of early stage tissue changes like for instance small tumours or metastases.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extracellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

WO-A-99/35508 discloses a method of MR investigation of a patient using a hyperpolarised solution of a high T₁ agent as MRI contrast agent. The term “hyperpolarisation” means enhancing the nuclear polarisation of NMR active nuclei present in the high T₁ agent, i.e. nuclei with non-zero nuclear spin, preferably ¹³C— or ¹⁵N-nuclei. Upon enhancing the nuclear polarisation of NMR active nuclei, the population difference between excited and ground nuclear spin states of these nuclei is significantly increased and thereby the MR signal intensity is amplified by a factor of hundred and more. When using a hyperpolarised ¹³C- and/or ¹⁵N-enriched high T₁ agent, there will be essentially no interference from background signals as the natural abundance of ¹³C and/or ¹⁵N is negligible and thus the image contrast will be advantageously high. The main difference between conventional MRI contrast agents and these hyperpolarised high T₁ agents is that in the former changes in contrast are caused by affecting the relaxation times of water protons in the body whereas the latter class of agents can be regarded as non-radioactive tracers, as the signal obtained arises solely from the agent.

A variety of possible high T₁ agents for use as MR imaging agents are disclosed in WO-A-99/35508, including non-endogenous and endogenous compounds. As examples of the latter intermediates in normal metabolic cycles are mentioned which are said to be preferred for imaging metabolic activity. By in vivo imaging of metabolic activity, information of the metabolic status of a tissue may be obtained and said information may for instance be used to discriminate between healthy and diseased tissue.

Pyruvate for instance is a compound that plays a role in the citric acid cycle and the conversion of hyperpolarised ¹³C-pyruvate to its metabolites hyperpolarised ¹³C-lactate, hyperpolarised ¹³C-bicarbonate and hyperpolarised ¹³C-alanine can be used for in vivo MR studying of metabolic processes in the human body. Hyperpolarised ¹³C-pyruvate may for instance be used as an MR imaging agent for in vivo tumour imaging as described in detail in WO-A-2006/011810 and for assessing the viability of myocardial tissue by MR imaging as described in detail in WO-A-2006/054903.

The metabolic conversion of hyperpolarised ¹³C-pyruvate to its metabolites hyperpolarised ¹³C-lactate, hyperpolarised ¹³C-bicarbonate and hyperpolarised ¹³C-alanine can be used for in vivo MR study of metabolic processes in the human body since said conversion has been found to be fast enough to allow signal detection from the parent compound, i.e. hyperpolarised ¹³C₁-pyruvate, and its metabolites. The amount of alanine, bicarbonate and lactate is dependent on the metabolic status of the tissue under investigation. The MR signal intensity of hyperpolarised ¹³C-lactate, hyperpolarised ¹³C-bicarbonate and hyperpolarised ¹³C-alanine is related to the amount of these compounds and the degree of polarisation left at the time of detection, hence by monitoring the conversion of hyperpolarised ¹³C-pyruvate to hyperpolarised ¹³C-lactate, hyperpolarised ¹³C-bicarbonate and hyperpolarised ¹³C-alanine it is possible to study metabolic processes in vivo in the human or non-human animal body by using non-invasive MR imaging or MR spectroscopy.

The MR signal amplitudes arising from the different pyruvate metabolites vary depending on the tissue type. The unique metabolic peak pattern formed by alanine, lactate, bicarbonate and pyruvate can be used as fingerprint for the metabolic state of the tissue under examination.

However, the production of hyperpolarised ¹³C-pyruvate which is suitable as an in vivo imaging agent is not without challenges. Hyperpolarised ¹³C-pyruvate is preferably obtained by dynamic nuclear polarisation (DNP) of either ¹³C-pyruvic acid or a ¹³C-pyruvate salt as described in detail in WO-A1-2006/011809, which is incorporated herein by reference.

The use of ¹³C-pyruvic acid simplifies the polarisation process since it does not crystallize upon freezing/cooling (crystallization leads to low dynamic nuclear polarisation or no polarisation at all). As a consequence no solvents and/or glass formers are needed to prepare a composition for the DNP process and thus a highly concentrated ¹³C-pyruvic acid sample can be used. However, due to its low pH a DNP agent needs to be used which is stable in the strong pyruvic acid. Further, a strong base is necessary to dissolve and convert the solid hyperpolarised ¹³C-pyruvic acid after the polarisation to hyperpolarised ¹³C-pyruvate. Both the strong pyruvic acid and the strong base require careful selection of materials (e.g. dissolution medium reservoir, tubes, etc.) the compounds get in contact with.

Alternatively, a ¹³C-pyruvate salt may be used in the DNP process. Unfortunately, sodium ¹³C-pyruvate crystallizes upon freezing/cooling which makes it necessary to add glass formers. If the hyperpolarised ¹³C-pyruvate is intended to be used as in vivo imaging agent, the pyruvate concentration in the composition containing the pyruvate and glass formers is unfavourably low. Besides, the glass formers are to be removed for in vivo use as well.

Thus preferred salts which may be used for DNP are those ¹³C-pyruvates which comprise an inorganic cation from the group consisting of NH₄⁺, K⁺, Rb⁺, Cs⁺, Ca²⁺, Sr²⁺ and Ba²⁺, preferably NH₄⁺, K⁺, Rb⁺ or Cs⁺, more preferably K⁺, Rb⁺, Cs⁺ and most preferably Cs⁺, as in detail described in WO-A-2007/111515. Most of these salts are not commercially available and need to be synthesized separately. Further, if the hyperpolarised ¹³C-pyruvate is used in vivo MR imaging it is preferred to exchange the inorganic cation from the group consisting of NH₄⁺, K⁺, Rb⁺, Cs⁺, Ca²⁺, Sr²⁺ and Ba²⁺ by a physiologically very well tolerable cation like Na⁺ or meglumine. Hence an additional step is required after liquefaction of the solid hyperpolarised ¹³C-pyruvate during which polarisation decays.

Other preferred salts are ¹³C-pyruvate of an organic amine or amino compound, preferably TRIS-¹³C₁-pyruvate or meglumine-¹³C₁-pyruvate, as in detail described in WO-A-2007/069909. Again these salts need to be synthesized separately.

We have now found that hyperpolarised ¹³C-lactate may be used as imaging agent in MR imaging and/or MR spectroscopy instead of hyperpolarised ¹³C-pyruvate.

Sodium ¹³C-lactate is a commercially available compound which may be directly used for DNP since it does not crystallize upon cooling/freezing. Since this eliminates the necessity of glass formers and/or high amounts of solvent(s) in the sample, a highly concentrated sample can be prepared and used in the DNP process. Further, sodium ¹³C-lactate samples are pH neutral and hence a variety of DNP agents can be used. Lactate is an endogenous compound and its concentration in human blood is fairly high (1-3 mM) with local concentrations of 10 mM and more. Hence, lactate is very well tolerated and using hyperpolarised ¹³C-lactate as an imaging agent is advantageous from a safety perspective.

Thus, in a first aspect the invention provides a method of ¹³C-MR detection using an imaging medium comprising hyperpolarised ¹³C-lactate.

The term “¹³C-MR detection” denotes ¹³C-MR imaging or ¹³C-MR spectroscopy or combined ¹³C-MR imaging and ¹³C-MR spectroscopy, i.e. ¹³C-MR spectroscopic imaging. The term further denotes ¹³C-MR spectroscopic imaging at various time points.

The term “imaging medium” denotes a liquid composition comprising hyperpolarised ¹³C-lactate as the MR active agent, i.e. imaging agent. The imaging medium according to the invention may be used as imaging medium in a method of ¹³C-MR detection.

The imaging medium used in the method of the invention may be used as an imaging medium for in vivo ¹³C-MR detection, i.e. in living human or non-human animal beings. Further, the imaging medium used in the method of the invention may be used as imaging medium for in vitro ¹³C-MR detection, e.g. in cell cultures, body samples like for instance urine, saliva or blood, ex vivo tissue, for instance ex vivo tissue obtained from a biopsy or isolated organs.

The terms “lactate” and “lactic acid”, unless specified otherwise, denote the L-isomer (L-lactate, L-lactic acid), the D-isomer (D-lactate, D-lactic acid) and mixtures of the L- and D-isomer (D/L-lactate and D/L-lactic acid), e.g. a racemic mixture of the D- and L-isomer. D-lactate and L-lactate are converted to pyruvate by different enzymes (i.e. D- and L-lactate dehydrogenase, respectively); however, the metabolites formed are pyruvate, lactate, alanine and bicarbonate for both of the isomers and hence both isomers can be used in the method of the invention.

The imaging medium according to the invention may thus comprise hyperpolarised ¹³C-L-lactate or hyperpolarised ¹³C-D-lactate or a mixture thereof, e.g. a racemic mixture of hyperpolarised ¹³C-D/L-lactate. In a preferred embodiment, the imaging medium according to the invention comprises hyperpolarised ¹³C-L-lactate or a mixture of hyperpolarised ¹³C-L-lactate and hyperpolarised ¹³C-D-lactate, more preferably a racemic mixture. In a most preferred embodiment, the imaging medium according to the invention comprises hyperpolarised ¹³C-L-lactate.

The term “¹³C-lactate” denotes a salt of ¹³C-lactic acid that is isotopically enriched with ¹³C, i.e. in which the amount of ¹³C isotope is greater than its natural abundance. Unless otherwise specified, the term “¹³C-lactate” and “¹³C-lactic acid” denote a compound which is ¹³C-enriched at any of the 3 carbon atoms present in the molecule, i.e. at the C1-position and/or the C2-position and/or the C3-position.

The isotopic enrichment of the hyperpolarised ¹³C-lactate used in the method of the invention is preferably at least 75%, more preferably at least 80% and especially preferably at least 90%, an isotopic enrichment of over 90% being most preferred. Ideally, the enrichment is 100%. ¹³C-lactate used in the method of the invention may be isotopically enriched at the C1-position (in the following denoted ¹³C₁-lactate), at the C2-position (in the following denoted ¹³C₂-lactate), at the C3-position (in the following denoted ¹³C₃-lactate), at the C1- and the C2-position (in the following denoted ¹³C_{1,2}-lactate), at the C1- and the C3-position (in the following denoted ¹³C_{1,3}-lactate), at the C2- and the C3-position (in the following denoted ¹³C_{2,3}-lactate) or at the C1-, C2- and C3-position (in the following denoted ¹³C_{1,2,3}-lactate). Isotopic enrichment at the C1-position is the most preferred since ¹³C₁-lactate has a higher, i.e. longer T₁ relaxation in human full blood at 37° C. than ¹³C-lactate which is isotopically enriched at other C-positions.

In a preferred embodiment, the imaging medium according to the invention comprises hyperpolarised sodium ¹³C-lactate, more preferably sodium ¹³C₁-lactate.

The terms “hyperpolarised” and “polarised” are used interchangeably hereinafter and denote a nuclear polarisation level in excess of 0.1%, more preferred in excess of 1% and most preferred in excess of 10%.

The level of polarisation may for instance be determined by solid state ¹³C-NMR measurements in solid hyperpolarised ¹³C-lactate, e.g. solid hyperpolarised ¹³C-lactate obtained by dynamic nuclear polarisation (DNP) of ¹³C-lactate. The solid state ¹³C-NMR measurement preferably consists of a simple pulse-acquire NMR sequence using a low flip angle. The signal intensity of the hyperpolarised ¹³C-lactate in the NMR spectrum is compared with signal intensity of ¹³C-lactate in a NMR spectrum acquired before the polarisation process. The level of polarisation is then calculated from the ratio of the signal intensities before and after polarisation.

In a similar way, the level of polarisation for dissolved hyperpolarised ¹³C-lactate may be determined by liquid state NMR measurements. Again the signal intensity of the dissolved hyperpolarised ¹³C-lactate is compared with the signal intensity of the dissolved ¹³C-lactate before polarisation. The level of polarisation is then calculated from the ratio of the signal intensities of ¹³C-lactate before and after polarisation.

Hyperpolarisation of NMR active ¹³C-nuclei may be achieved by different methods which are for instance described in described in WO-A-98/30918, WO-A-99/24080 and WO-A-99/35508, and which all are incorporated herein by reference and hyperpolarisation methods known in the art are polarisation transfer from a noble gas, “brute force”, spin refrigeration, the parahydrogen method and dynamic nuclear polarisation (DNP).

To obtain hyperpolarised ¹³C-lactate, it is preferred to polarise ¹³C-lactate directly. Also ¹³C-lactic acid may be polarised, however the polarised ¹³C-lactic acid needs to be converted to polarised ¹³C-lactate, e.g. by neutralisation with a base. ¹³C-lactate salts are commercially available, e.g. sodium ¹³C-lactate. ¹³C-lactic acid is commercially available as well; it can also be obtained by protonating commercially available ¹³C-lactate, e.g. commercially available sodium ¹³C-lactate.

One way for obtaining hyperpolarised ¹³C-lactate is the polarisation transfer from a hyperpolarised noble gas which is described in WO-A-98/30918. Noble gases having non-zero nuclear spin can be hyperpolarised by the use of circularly polarised light. A hyperpolarised noble gas, preferably He or Xe, or a mixture of such gases, may be used to effect hyperpolarisation of ¹³C-nuclei. The hyperpolarised gas may be in the gas phase, it may be dissolved in a liquid/solvent, or the hyperpolarised gas itself may serve as a solvent. Alternatively, the gas may be condensed onto a cooled solid surface and used in this form, or allowed to sublime. Intimate mixing of the hyperpolarised gas with ¹³C-lactate or ¹³C-lactic acid is preferred.

Another way for obtaining hyperpolarised ¹³C-lactate is that polarisation is imparted to ¹³C-nuclei by thermodynamic equilibration at a very low temperature and high field. Hyperpolarisation compared to the operating field and temperature of the NMR spectrometer is effected by use of a very high field and very low temperature (brute force). The magnetic field strength used should be as high as possible, suitably higher than 1 T, preferably higher than 5 T, more preferably 15 T or more and especially preferably 20 T or more. The temperature should be very low, e.g. 4.2 K or less, preferably 1.5 K or less, more preferably 1.0 K or less, especially preferably 100 mK or less.

Another way for obtaining hyperpolarised ¹³C-lactate is the spin refrigeration method. This method covers spin polarisation of a solid compound or system by spin refrigeration polarisation. The system is doped with or intimately mixed with suitable crystalline paramagnetic materials such as Ni²⁺, lanthanide or actinide ions with a symmetry axis of order three or more. The instrumentation is simpler than required for DNP with no need for a uniform magnetic field since no resonance excitation field is applied. The process is carried out by physically rotating the sample around an axis perpendicular to the direction of the magnetic field. The pre-requisite for this method is that the paramagnetic species has a highly anisotropic g-factor. As a result of the sample rotation, the electron paramagnetic resonance will be brought in contact with the nuclear spins, leading to a decrease in the nuclear spin temperature. Sample rotation is carried out until the nuclear spin polarisation has reached a new equilibrium.

In a preferred embodiment, DNP (dynamic nuclear polarisation) is used to obtain hyperpolarised ¹³C-lactate. In DNP, polarisation of MR active nuclei in a compound to be polarised is affected by a polarisation agent or so-called DNP agent, a compound comprising unpaired electrons. During the DNP process, energy, normally in the form of microwave radiation, is provided, which will initially excite the DNP agent. Upon decay to the ground state, there is a transfer of polarisation from the unpaired electron of the DNP agent to the NMR active nuclei of the compound to be polarised, e.g. to the ¹³C nuclei in ¹³C-lactate. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. The DNP technique is for example further described in WO-A-98/58272 and in WO-A-01/96895, both of which are included by reference herein.

To polarise a chemical entity, i.e. compound, by the DNP method, a composition comprising the compound to be polarised and a DNP agent is prepared which is then frozen and inserted into a DNP polariser for polarisation. After the polarisation, the frozen solid hyperpolarised composition is rapidly transferred into the liquid state either by melting it or by dissolving it in a suitable dissolution medium. Dissolution is preferred and the dissolution process of a frozen hyperpolarised composition and suitable devices therefore are described in detail in WO-A-02/37132. The melting process and suitable devices for the melting are for instance described in WO-A-02/36005.

In order to obtain a high polarisation level in the compound to be polarised said compound and the DNP agent need to be in intimate contact during the DNP process. This is not the case if the composition crystallizes upon being frozen or cooled. To avoid crystallization, either glass formers need to be present in the composition or compounds need to be chosen for polarisation which do not crystallize upon being frozen but rather form a glass. Sodium ¹³C-lactate is especially preferred since compositions containing sodium ¹³C-lactate do not crystallize upon freezing/cooling.

In one embodiment, ¹³C-lactic acid, preferably ¹³C₁-lactic acid is used as a starting material to obtain hyperpolarised ¹³C-lactate by the DNP method. Said ¹³C-lactic acid may be ¹³C-L-lactic acid, ¹³C-D-lactic acid or a mixture thereof, e.g. a racemic mixture of ¹³C-D/L-lactic acid. In a preferred embodiment, said ¹³C-lactic acid is ¹³C-L-lactic acid or a mixture of ¹³C-L-lactic acid and ¹³C-D-lactic acid, more preferably a racemic mixture. In a most preferred embodiment, said ¹³C-lactic acid is ¹³C-L-lactic acid.

In a preferred embodiment, ¹³C-lactate, preferably ¹³C₁-lactate is used as a starting material to obtain hyperpolarised ¹³C-lactate by the DNP method. Said ¹³C-lactate may be ¹³C-L-lactate, ¹³C-D-lactate or a mixture thereof, e.g. a racemic mixture of ¹³C-D/L-lactate. In a preferred embodiment, said ¹³C-lactate is ¹³C-L-lactate or a mixture of ¹³C-L-lactate and ¹³C-D-lactate, more preferably a racemic mixture. In a most preferred embodiment, said ¹³C-lactate is ¹³C-L-lactate. Suitable ¹³C-lactates are sodium ¹³C-lactate and ¹³C-lactates which comprise an inorganic cation from the group consisting of NH₄⁺, K⁺, Rb⁺, Cs⁺, Ca²⁺, Sr²⁺ and Ba²⁺. The latter salts are described in detail in WO-A-2007/111515 which is incorporated by reference herein. Alternatively, ¹³C-lactates of an organic amine or amino compound, preferably TRIS-¹³C-lactate or meglumine-¹³C-lactate, as in detail described in WO-A-2007/069909 and incorporated by reference herein. In a most preferred embodiment sodium ¹³C-lactate and more preferably sodium ¹³C₁-lactate and most preferably sodium ¹³C₁-L-lactate is used as a starting material to obtain hyperpolarised lactate by the DNP method.

For the hyperpolarisation of ¹³C-lactate by DNP, a composition is prepared which comprises C-lactate or ¹³C-lactic acid and a DNP agent.

The DNP agent plays a decisive role in the DNP process as its choice has a major impact on the level of polarisation that can be achieved in ¹³C-lactate. A variety of DNP agents—in WO-A-99/35508 denoted “OMRI contrast agents” —is known. The use of oxygen-based, sulphur-based or carbon-based stable trityl radicals as described in WO-A-99/35508, WO-A-88/10419, WO-A-90/00904, WO-A-91/12024, WO-A-93/02711 or WO-A-96/39367 has resulted in high levels of polarisation in a variety of different samples.

In a preferred embodiment, the hyperpolarised ¹³C-lactate used in the method of the invention is obtained by DNP and the DNP agent used is a trityl radical. As briefly mentioned above, the large electron spin polarisation of the DNP agent, i.e. trityl radical is converted to nuclear spin polarisation of ¹³C nuclei in ¹³C-lactate or ¹³C-lactic acid via microwave irradiation close to the electron Larmor frequency. The microwaves stimulate communication between electron and nuclear spin systems via e-e and e-n transitions. For effective DNP, i.e. to achieve a high level of polarisation in ¹³C-lactate or ¹³C-lactic acid the trityl radical has to be stable and soluble in these compounds to achieve intimate contact between ¹³C-lactate/¹³C-lactic acid and the trityl radical which is necessary for the aforementioned communication between electron and nuclear spin systems.

In a preferred embodiment, the trityl radical is a radical of the formula (1)

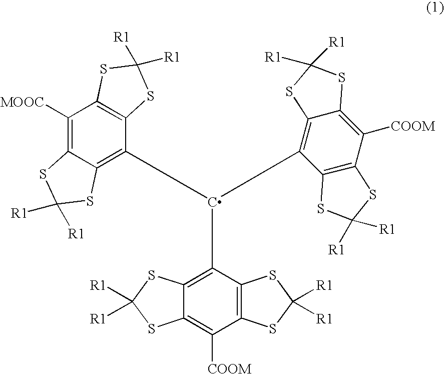

wherein
M represents hydrogen or one equivalent of a cation; and
R1 which is the same or different represents a straight chain or branched C₁-Cₒ-alkyl group optionally substituted by one or more hydroxyl groups or a group —(CH₂)ₙ—X—R2,
   wherein n is 1, 2 or 3;
   X is O or S; and
   R2 is a straight chain or branched C₁-C₄-alkyl group, optionally substituted by one or more hydroxyl groups.

In a preferred embodiment, M represents hydrogen or one equivalent of a physiologically tolerable cation. The term “physiologically tolerable cation” denotes a cation that is tolerated by the human or non-human animal living body. Preferably, M represents hydrogen or an alkali cation, an ammonium ion or an organic amine ion, for instance meglumine. Most preferably, M represents hydrogen or sodium.

If ¹³C-lactate is used as a starting material to obtain hyperpolarised ¹³C-lactate by the DNP method, R1 is preferably the same, more preferably a straight chain or branched C₁-C₄-alkyl group, most preferably methyl, ethyl or isopropyl; or R1 is preferably the same, more preferably a straight chain or branched C₁-C₄-alkyl group which is substituted by one hydroxyl group, most preferably —CH₂—CH₂—OH; or R1 is preferably the same and represents —CH₂—OC₂H₄OH.

If ¹³C-lactic acid is used as a starting material to obtain hyperpolarised ¹³C-lactate by the DNP method, R1 is the same or different, preferably the same and preferably represents —CH₂—OCH₃, —CH₂—OC₂H₅, —CH₂—CH₂—OCH₃, —CH₂—SCH₃, —CH₂—SC₂H₅ or —CH₂—CH₂—SCH₃, most preferably —CH₂—CH₂—OCH₃.

The aforementioned trityl radical of formula (1) may be synthesized as described in detail in WO-A-88/10419, WO-A-90/00904, WO-A-91/12024, WO-A-93/02711, WO-A-96/39367, WO-A-97/09633, WO-A-98/39277 and WO-A-2006/011811.

For the DNP process, a solution of the starting material ¹³C-lactic acid or ¹³C-lactate (in the following denoted “sample”) and the DNP agent, preferably a trityl radical, more preferably a trityl radical of formula (1) is prepared. A solvent or a solvent mixture may be used to promote dissolution of the DNP agent in the sample. However, if the hyperpolarised ¹³C-lactate is intended to be used as an imaging agent for in vivo ¹³C-MR detection, it is preferred to keep the amount of solvent to a minimum or, if possible, to avoid the use of solvents. To be used as an in vivo imaging agent, the polarised ¹³C-lactate is usually administered in relatively high concentrations, i.e. a highly concentrated sample is preferably used in the DNP process and hence the amount of solvent is preferably kept to a minimum. In this context, it is also important to mention that the mass of the composition containing the sample, i.e. DNP agent, sample and if necessary solvent, is kept as small as possible. A high mass will have a negative impact on the efficiency of the dissolution process, if dissolution is used to convert the solid composition containing the hyperpolarised ¹³C-lactic acid or ¹³C-lactate after the DNP process into the liquid state, e.g. for using it as an imaging agent for ¹³C-MR detection. This is due to the fact that for a given volume of dissolution medium in the dissolution process, the mass of the composition to dissolution medium ratio decreases, when the mass of the composition increases. Further, using certain solvents may require their removal before the hyperpolarised ¹³C-lactate used as an MR imaging agent is administered to a human or non-human animal being since they might not be physiologically tolerable.

If ¹³C-lactic acid is used as a starting material to obtain hyperpolarised ¹³C-lactate via DNP, preferably a solution of the DNP agent, preferably a trityl radical and more preferably a trityl radical of formula (1) in ¹³C-lactic acid is prepared. Mixtures of ¹³C-L-lactic acid and ¹³C-D-lactic acid are either liquids at room temperature (the ¹³C-D/L-lactic acid racemic mixture has a melting point of about 17° C.) or have a melting point which is between the melting point of the pure isomer and the racemate, i.e. between 17° C.-53° C. If a mixture of ¹³C-L-lactic acid and ¹³C-D-lactic acid is used which is a liquid at room temperature, the DNP agent is preferably dissolved in said liquid without further addition of any solvents. However, if solvent(s) are added, it is preferred to use a solvent which is a good glass former, e.g. glycerol. If a mixture of ¹³C-L-lactic acid and ¹³C-D-lactic acid is used or if ¹³C-L-lactic acid or ¹³C-D-lactic acid are used (both have a melting point of about 53° C.), this mixture or the ¹³C-L-lactic acid or ¹³C-D-lactic acid are preferably melted under gentle warming and the DNP agent is dissolved in the melted mixture or ¹³C-L-lactic acid or ¹³C-D-lactic acid. Preferably, no solvents are added. However, if solvent(s) are added, it is preferred to either add little water and/or add a solvent which is a good glass former, e.g. glycerol. Intimate mixing of the compounds can be promoted by several means known in the art, such as stirring, vortexing (whirl-mixing) or sonication.

If a ¹³C-lactate which is a solid at room temperature is used as a starting material to obtain hyperpolarised ¹³C-lactate via DNP, a solvent has to be added to prepare a solution of the DNP agent and the ¹³C-lactate. Preferably an aqueous carrier and most preferably water is used as a solvent. In one embodiment, the DNP agent is dissolved and the ¹³C-lactate is subsequently dissolved in the dissolved DNP agent. In another embodiment, ¹³C-lactate is dissolved in the solvent and subsequently the DNP agent is dissolved in the dissolved ¹³C-lactate. If the ¹³C-lactates mentioned in the first paragraph on page 10, i.e. sodium ¹³C-lactate, ¹³C-lactates which comprise an inorganic cation from the group consisting of NH₄⁺, K⁺, Rb⁺, Cs⁺, Ca²⁺, Sr²⁺ and Ba²⁺ and ¹³C-lactates of an organic amine or amino compound are used, no glass formers have to be added, since a composition containing these ¹³C-lactates does not crystallize upon cooling/freezing. Again intimate mixing of the compounds can be promoted by several means known in the art, such as stirring, vortexing or sonication.

If the hyperpolarised ¹³C-lactate used in the method of the invention is obtained by DNP, the composition to be polarised comprising ¹³C-lactic acid or ¹³C-lactate and a DNP agent may further comprise a paramagnetic metal ion. It has been found that the presence of paramagnetic metal ions may result in increased polarisation levels in the compound to be polarised by DNP as described in detail in WO-A2-2007/064226 which is incorporated herein by reference. The term “paramagnetic metal ion” denotes paramagnetic metal ions in the form of their salts and paramagnetic chelates, i.e. chemical entities comprising a chelator and a paramagnetic metal ion, wherein said paramagnetic metal ion and said chelator form a complex.

In a preferred embodiment, the paramagnetic metal ion is a compound comprising Gd³⁺ as a paramagnetic metal ion, preferably a paramagnetic chelate comprising a chelator and Gd³⁺ as a paramagnetic metal ion. In a more preferred embodiment, said paramagnetic metal ion is soluble and stable in the composition to be polarised.

As with the DNP agent described before, the ¹³C-lactic acid or ¹³C-lactate to be polarised must be in intimate contact with the paramagnetic metal ion as well. The composition used for DNP comprising ¹³C-lactic acid or ¹³C-lactate, a DNP agent and a paramagnetic metal ion may be obtained in several ways. In a first embodiment the ¹³C-lactate is dissolved in a suitable solvent to obtain a solution; alternatively, liquid or melted ¹³C-lactic acid as discussed on the previous page is used. To this solution of ¹³C-lactate or to the liquid/melted ¹³C-lactic acid the DNP agent is added and dissolved. The DNP agent, preferably a trityl radical, might be added as a solid or in solution, preferably as a solid. In a subsequent step, the paramagnetic metal ion is added. The paramagnetic metal ion might be added as a solid or in solution, preferably as a solid. In another embodiment, the DNP agent and the paramagnetic metal ion are dissolved in a suitable solvent this solution is added to ¹³C-lactic acid or ¹³C-lactate. In yet another embodiment, the DNP agent (or the paramagnetic metal ion) is dissolved in a suitable solvent and added to ¹³C-lactic acid or ¹³C-lactate. In a subsequent step the paramagnetic metal ion (or the DNP agent) is added to this solution, either as a solid or in solution, preferably as a solid. Preferably, the amount of solvent to dissolve the paramagnetic metal ion (or the DNP agent) is kept to a minimum. Again intimate mixing of the compounds can be promoted by several means known in the art, such as stirring, vortexing or sonication.

If a trityl radical is used as DNP agent, a suitable concentration of such a trityl radical in the composition is 1 to 25 mM, preferably 2 to 20 mM, more preferably 10 to 15 mM in the composition used for DNP. If a paramagnetic metal ion is added to the composition, a suitable concentration of such a paramagnetic metal ion is 0.1 to 6 mM (metal ion) in the composition, and a concentration of 0.5 to 4 mM is preferred.

After having prepared a composition comprising ¹³C-lactic acid or ¹³C-lactate, the DNP agent and optionally a paramagnetic metal ion said composition is frozen by methods known in the art, e.g. by freezing it in a freezer, in liquid nitrogen or by simply placing it in the DNP polariser, where liquid helium will freeze it. The composition may optionally be frozen as “beads” before it is inserted into to polariser. Such beads may be obtained by adding the composition drop wise to liquid nitrogen. A more efficient dissolution of such beads has been observed, which is especially relevant if larger amounts of ¹³C-lactic acid or ¹³C-lactate are polarised, for instance when it is intended to use the polarised ¹³C-lactate in an in vivo ¹³C-MR detection method.

If a paramagnetic metal ion is present in the composition said composition may be degassed before freezing, e.g. by bubbling helium gas through the composition (for instance for a time period of 2-15 min) but degassing can be effected by other known common methods.

The DNP technique is for instance described in WO-A-98/58272 and in WO-A-01/96895, both of which are included by reference herein. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. In a preferred embodiment, the DNP process is carried out in liquid helium and a magnetic field of about 1 T or above. Suitable polarisation units are for instance described in WO-A-02/37132. In a preferred embodiment, the polarisation unit comprises a cryostat and polarising means, e.g. a microwave chamber connected by a wave guide to a microwave source in a central bore surrounded by magnetic field producing means such as a superconducting magnet. The bore extends vertically down to at least the level of a region P near the superconducting magnet where the magnetic field strength is sufficiently high, e.g. between 1 and 25 T, for polarisation of the sample nuclei to take place. The bore for the probe (i.e. the frozen composition to be polarised) is preferably sealable and can be evacuated to low pressures, e.g. pressures in the order of 1 mbar or less. A probe introducing means such as a removable transporting tube can be contained inside the bore and this tube can be inserted from the top of the bore down to a position inside the microwave chamber in region P. Region P is cooled by liquid helium to a temperature low enough to for polarisation to take place, preferably temperatures of the order of 0.1 to 100 K, more preferably 0.5 to 10 K, most preferably 1 to 5 K. The probe introducing means is preferably sealable at its upper end in any suitable way to retain the partial vacuum in the bore. A probe-retaining container, such as a probe-retaining cup, can be removably fitted inside the lower end of the probe introducing means. The probe-retaining container is preferably made of a light-weight material with a low specific heat capacity and good cryogenic properties such, e.g. KelF (polychlorotrifluoro-ethylene) or PEEK (polyetheretherketone) and it may be designed in such a way that it can hold more than one probe.

The probe is inserted into the probe-retaining container, submerged in the liquid helium and irradiated with microwaves. The microwave frequency may be determined from the EPR line of the DNP agent, which depends on the magnetic field of the magnet as 28.0 GHz/T. The optimal microwave frequency may be determined by adjusting the frequency for maximal NMR signal. Preferably, the optimal microwave frequency is in the about 94 GHz for a magnet charged to 3.35 T, 110 GHz for a magnet charged to 4 T, 140 GHz for a magnet charged to 5 T and 200 GHz for a magnet charged to 7 T. The power may be chosen between 50 and 200 mW, dependent on the probe size. The level of polarisation may be monitored as earlier described by for instance acquiring solid state ¹³C-NMR signals of the probe during microwave irradiation. Generally, a saturation curve is obtained in a graph showing NMR signal vs. time. Hence it is possible to determine when the optimal polarisation level is reached. A solid state ¹³C-NMR measurement suitably consists of a simple pulse-acquire NMR sequence using a low flip angle. The signal intensity of the dynamic nuclear polarised nuclei, i.e. ¹³C nuclei in ¹³C-lactic acid or ¹³C-lactate is compared with the signal intensity of the ¹³C nuclei in ¹³C-lactic acid or ¹³C-lactate before DNP. The polarisation is then calculated from the ratio of the signal intensities before and after DNP.

After the DNP process, the frozen solid composition comprising the hyperpolarised ¹³C-lactic acid or ¹³C-lactate is transferred from the solid state to the liquid state, i.e. liquefied. This can be done by dissolution in an appropriate solvent or solvent mixture (dissolution medium) or by melting the solid composition, e.g. by applying energy in the form of heat. Dissolution is preferred and the dissolution process and suitable devices therefore are described in detail in WO-A-02/37132. The melting process and suitable devices for the melting are for instance described in WO-A-02/36005. Briefly, a dissolution unit/melting unit is used which is either physically separated from the polariser or is a part of an apparatus that contains the polariser and the dissolution unit/melting unit. In a preferred embodiment, dissolution/melting is carried out at an elevated magnetic field, e.g. inside the polariser, to improve the relaxation and retain a maximum of the hyperpolarisation. Field nodes should be avoided and low field may lead to enhanced relaxation despite the above measures.

If ¹³C-lactate has been used as the starting material for the dynamic nuclear polarisation and if the solid composition comprising the hyperpolarised ¹³C-lactate is liquefied by dissolution, an aqueous carrier, preferably a physiologically tolerable and pharmaceutically accepted aqueous carrier like water, a buffer solution or saline is suitably used as a solvent especially preferably if the hyperpolarised ¹³C-lactate is intended for use in an imaging medium for in vivo ¹³C-MR detection. For in vitro applications also non aqueous solvents or solvent mixtures may be used for instance DMSO or methanol or mixtures comprising an aqueous carrier and a non aqueous solvent, for instance mixtures of DMSO and water or methanol and water.

If ¹³C-lactic acid has been used as the starting material for the dynamic nuclear polarisation, the hyperpolarised ¹³C-lactic acid obtained has to be converted to ¹³C-lactate. If the solid composition comprising the hyperpolarised ¹³C-lactic acid is liquefied by dissolution, the dissolution medium preferably is an aqueous carrier, e.g. water or a buffer solution, preferably a physiologically tolerable buffer solution or comprises an aqueous carrier, e.g. water or a buffer solution, preferably a physiologically tolerable buffer solution. The terms “buffer solution” and “buffer” are hereinafter used interchangeably. In the context of this application “buffer” denotes one or more buffers, i.e. also mixtures of buffers.

Preferred buffers are physiologically tolerable buffers, more preferably buffers which buffer in the range of about pH 7 to 8 like for instance phosphate buffer (KH₂PO₄/Na₂HPO₄), ACES, PIPES, imidazole/HCl, BES, MOPS, HEPES, TES, TRIS, HEPPS or TRICIN.

To convert hyperpolarised ¹³C-lactic acid into hyperpolarised ¹³C-lactate, ¹³C-lactic acid is suitably reacted with a base. In one embodiment, ¹³C-lactic acid is reacted with a base to convert it to ¹³C-lactate and subsequently an aqueous carrier is added. In another preferred embodiment the aqueous carrier and the base are combined in one solution and this solution is added to ¹³C-lactic acid, dissolving it and converting it into ¹³C-lactate at the same time. In a preferred embodiment, the base is an aqueous solution of NaOH, Na₂CO₃ or NaHCO₃, most preferred the base is an aqueous solution of NaOH.

In another preferred embodiment, the aqueous carrier or—where applicable—the combined aqueous carrier/base solution further comprises one or more compounds which are able to bind or complex free paramagnetic ions, e.g. chelating agents like DTPA or EDTA.

If hyperpolarisation is carried out by the DNP method, the DNP agent, preferably a trityl radical and the optional paramagnetic metal ion may be removed from the liquid containing the hyperpolarised ¹³C-lactate. Removal of these compounds is preferred if the hyperpolarised ¹³C-lactate is intended for use in an imaging medium for in vivo use. If ¹³C-lactic acid was as a starting material for DNP, it is preferred to first convert the hyperpolarised ¹³C-lactic acid into ¹³C-lactate and remove the DNP agent and the optional paramagnetic metal ion after the conversion has taken place.

Methods useful to remove the trityl radical and the paramagnetic metal ion are known in the art and described in detail in WO-A2-2007/064226 and WO-A1-2006/011809.

In a preferred embodiment the hyperpolarised ¹³C-lactate used in the method of the invention is obtained by dynamic nuclear polarisation of a composition that comprises sodium ¹³C-lactate, preferably sodium ¹³C₁-lactate and more preferably sodium ¹³C₁-L-lactate, a trityl radical of formula (1) and optionally a paramagnetic chelate comprising Gd³⁺. In this preferred embodiment, a solution of the trityl radical and, if used, the paramagnetic chelate comprising Gd³⁺ is prepared. The dissolved trityl radical and the optional dissolved paramagnetic chelate are added to sodium ¹³C-lactate and the composition is preferably sonicated or whirl-mixed to promote intimate mixing of all the components.

The imaging medium according to the method of the invention may be used as imaging medium for in vitro ¹³C-MR detection, e.g. ¹³C-MR detection in cell cultures, body samples, ex vivo tissue or isolated organs derived from the human or non-human animal body. For this purpose, the imaging medium is provided as a composition that is suitable for being added to, for instance, cell cultures, samples like urine, blood or saliva, ex vivo tissues like biopsy tissues or isolated organs. Such an imaging medium preferably comprises in addition to the imaging agent, i.e. the MR active agent hyperpolarised ¹³C-lactate a solvent which is compatible with and used for in vitro cell or tissue assays, for instance DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such an imaging medium but are not required for such a purpose.

Further, the imaging medium according to the method of the invention may be used as imaging medium for in vivo ¹³C-MR detection, i.e. ¹³C-MR detection carried out on living human or non-human animal beings. For this purpose, the imaging medium needs to be suitable for administration to a living human or non-human animal body. Hence such an imaging medium preferably comprises in addition to the imaging agent, i.e. the MR active agent hyperpolarised ¹³C-lactate, an aqueous carrier, preferably a physiologically tolerable and pharmaceutically accepted aqueous carrier like water, a buffer solution or saline. Such an imaging medium may further comprise conventional pharmaceutical or veterinary carriers or excipients, e.g. formulation aids such as stabilizers, osmolality adjusting agents, solubilising agents and the like which are conventional for diagnostic compositions in human or veterinary medicine.

If the imaging medium used in the method of the invention is used for in vivo ¹³C-MR detection, i.e. in a living human or non-human animal body, said imaging medium is preferably administered to said body parenterally, preferably intravenously. Generally, the body under examination is positioned in an MR magnet. Dedicated ¹³C-MR RF-coils are positioned to cover the area of interest. Dosage and concentration of the imaging medium will depend upon a range of factors such as toxicity and the administration route. At less than 400 s after the administration, preferably less than 120 s, more preferably less than 60 s after the administration, especially preferably 20 to 50 s an MR imaging sequence is applied that encodes the volume of interest in a combined frequency and spatial selective way. The exact time of applying an MR sequence is highly dependent on the volume of interest and on the species.

In the ¹³C-MR detection method according to the invention, it is preferred to detect signals of ¹³C-lactate, ¹³C-pyruvate, ¹³C-alanine and ¹³C-bicarbonate. The MR detectable ¹³C-labelled compounds are identical when either hyperpolarised ¹³C-lactate or hyperpolarised ¹³C-pyruvate is used as imaging agent. This is shown for ¹³C₁-lactate and ¹³C₁-pyruvate in scheme 1, wherein * denotes the ¹³C-label: on the left of scheme 1, the MR detectable signals of hyperpolarised ¹³C₁-pyruvate (bold, parent compound) and its metabolites ¹³C-lactate, ¹³C-alanine and ¹³C-bicarbonate are shown; on the right of scheme 1, the MR detectable signals of hyperpolarised ¹³C₁-lactate (bold, parent compound) and its metabolite ¹³C-pyruvate are shown. The latter further metabolizes to ¹³C-alanine and ¹³C-bicarbonate.

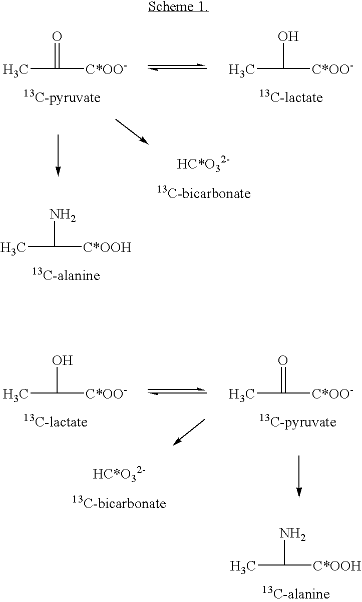

Thus in a preferred embodiment it is provided a method of ¹³C-MR detection using an imaging medium comprising hyperpolarised ¹³C-lactate, wherein signals of ¹³C-lactate, ¹³C-pyruvate and ¹³C-alanine, preferably signals of ¹³C-lactate, ¹³C-pyruvate, ¹³C-alanine and ¹³C-bicarbonate are detected.

The term “signal” in the context of the invention refers to the MR signal amplitude or integral or peak area to noise of peaks in a ¹³C-MR spectrum which represent ¹³C-lactate, ¹³C-pyruvate, ¹³C-alanine or ¹³C-bicarbonate. In a preferred embodiment, the signal is the peak area.

In a preferred embodiment of the method of the invention, the above-mentioned signals of ¹³C-lactate, ¹³C-pyruvate, ¹³C-alanine and ¹³C-bicarbonate are used to generate a metabolic profile.

In embodiment, the above-mentioned signals of ¹³C-lactate, ¹³C-pyruvate, ¹³C-alanine and ¹³C-bicarbonate are used to generate a metabolic profile of a living human or non-human animal being. Said metabolic profile may be derived from the whole body, e.g. obtained by whole body in vivo ¹³C-MR detection. Alternatively, said metabolic profile is generated from a region of interest, i.e. a certain tissue, organ or part of said human or non-human animal body.

In another embodiment, the above-mentioned signals of ¹³C-lactate, ¹³C-pyruvate, ¹³C-alanine and ¹³C-bicarbonate are used to generate a metabolic profile of cells in a cell culture, of samples like urine, blood or saliva, of ex vivo tissue like biopsy tissue or of an isolated organ. Said metabolic profile is then generated by in vitro ¹³C-MR detection.

Thus in a preferred embodiment it is provided a method of ¹³C-MR detection using an imaging medium comprising hyperpolarised ¹³C-lactate, wherein signals of ¹³C-lactate, ¹³C-pyruvate and ¹³C-alanine, preferably signals of ¹³C-lactate, ¹³C-pyruvate, ¹³C-alanine and ¹³C-bicarbonate are detected and wherein said signals are used to generate a metabolic profile.

Suitably, the signals of ¹³C-lactate, ¹³C-pyruvate and ¹³C-alanine are used to generate said metabolic profile. In a preferred embodiment, the signals of ¹³C-lactate, ¹³C-pyruvate, ¹³C-alanine and ¹³C-bicarbonate are used to generate a metabolic profile. Hereinafter the term “¹³C-labelled compounds” is used to denote ¹³C-lactate and ¹³C-pyruvate and ¹³C-alanine and to denote the preferred embodiment ¹³C-lactate and ¹³C-pyruvate and ¹³C-alanine and ¹³C-bicarbonate.

In one embodiment, the spectral signal intensities of the ¹³C-labelled compounds are used to generate the metabolic profile. In another embodiment, the spectral signal integrals of the ¹³C-labelled compounds are used to generate the metabolic profile. In another embodiment, signal intensities from separate images of the ¹³C-labelled compounds are used to generate the metabolic profile. In yet another embodiment, the signal intensities of the ¹³C-labelled compounds are obtained at two or more time points to calculate the rate of change of the ¹³C-labelled compounds.

In another embodiment the metabolic profile includes or is generated using processed signal data of the ¹³C-labelled compounds, e.g. ratios of signals, corrected signals, or dynamic or metabolic rate constant information deduced from the signal pattern of multiple MR detections, i.e. spectra or images. Thus, in a preferred embodiment a corrected ¹³C-lactate signal, i.e. ¹³C-lactate to ¹³C-alanine signal and/or ¹³C-lactate to ¹³C-pyruvate signal and/or ¹³C-lactate to ¹³C-bicarbonate signal is included into or used to generate the metabolic profile. In a further preferred embodiment, a corrected ¹³C-lactate to total ¹³C-carbon signal is included into or used to generate the metabolic profile with the total ¹³C-carbon signal being the sum of the signals of ¹³C-lactate, ¹³C-pyruvate, ¹³C-alanine and optionally ¹³C-bicarbonate.

The metabolic profile generated in the preferred embodiment of the method according to the invention provides information about the metabolic status and activity of the body, part of the body, cells, tissue, body sample etc under examination and said information may be used in a subsequent step for, e.g. identifying diseases, monitoring the course of a disease and/or determining a disease state or for monitoring therapy success.

Such a disease may be a tumour since tumour tissue is usually characterized by a higher metabolic activity than healthy tissue. Such a higher metabolic activity can be determined by comparing the metabolic profile of a tumour or of an ex vivo sample of a tumour with the metabolic profile of healthy tissue (e.g. surrounding tissue or healthy ex vivo tissue) and may manifest itself in said metabolic profile by high signals of the ¹³C-labelled compounds or high corrected ¹³C-lactate signal or high metabolic rates.

Another disease may be ischemia in the heart since ischemic myocardial tissue is usually characterized by a lower metabolic activity than healthy myocardial tissue. Again such a lower metabolic activity can be determined by comparing the metabolic profile of ischemic myocardial tissue with the metabolic profile of healthy myocardial tissue.

Yet another disease may be liver related diseases, such as liver fibrosis or liver cirrhosis. 60% of all lactate metabolism occurs in the liver and it is expected that due to cell death in liver diseases the signal of the ¹³C-labelled lactate metabolites will decrease in diseased areas of the liver. Thus a metabolic profile of a diseased liver would show a significantly decrease of signals from ¹³C-alanine and optionally from ¹³C-pyruvate or high corrected ¹³C-alanine signal or high ratio of ¹³C-alanine to ¹³C-lactate or total carbon.

If D-lactate is used in the method of the invention, diseases like sepsis, ischemia and diabetes and conditions like trauma may be identified (see for instance S. M. Smith et al., J, Infect. Dis. 154, (1986), 658-664; M. J. Murray et al., Am. J. Surg. 167, (1994), 575-578; Z. Li et al., Chin. Med. Sci. J. 16, (2001), 209-213 and Y. Kondoh et al., Res. Exp. Med 192, (1992), 407-414.

Yet another aspect of the invention is a composition comprising sodium ¹³C₁-lactate or ¹³C₁-lactic acid, a trityl radical and optionally a paramagnetic metal ion.

In a first embodiment, said composition comprises sodium ¹³C₁-lactate, a trityl radical and optionally a paramagnetic metal ion. In a preferred embodiment, said sodium ¹³C₁-lactate is ¹³C₁-L-lactate. In another preferred embodiment, said trityl radical is a trityl radical of formula (1) wherein M represents hydrogen or sodium and R1 is preferably the same, more preferably a straight chain or branched C₁-C₄-alkyl group, most preferably methyl, ethyl or isopropyl; or R1 is preferably the same, more preferably a straight chain or branched C₁-C₄-alkyl group which is substituted by one hydroxyl group, most preferably —CH₂—CH₂—OH; or R1 is preferably the same and represents —CH₂—OC₂H₄OH.

In another preferred embodiment said composition comprises a paramagnetic metal ion, and said paramagnetic metal ion is preferably a compound comprising Gd³⁺ as a paramagnetic metal ion, preferably a paramagnetic chelate comprising a chelator and Gd³⁺ as a paramagnetic metal ion. In a most preferred embodiment, the composition according to the invention comprises sodium ¹³C₁-L-lactate, a trityl radical of formula (1) and a paramagnetic metal ion. Suitably, said composition further comprises a solvent or solvents; preferably an aqueous carrier and most preferably water is used as a solvent. The aforementioned compositions can be used for obtaining hyperpolarised sodium ¹³C₁-lactate by dynamic nuclear polarisation (DNP) with a high polarisation level. In a second embodiment said composition comprises ¹³C₁-lactic acid, a trityl radical and optionally a paramagnetic metal ion. In a preferred embodiment, said ¹³C₁-lactic acid ¹³C₁-L-lactic acid. In another preferred embodiment, said trityl radical is a trityl radical of formula (1) wherein M represents hydrogen or sodium and R1 is the same or different, preferably the same and preferably represents —CH₂—OCH₃, —CH₂—OC₂H₅, —CH₂—CH₂—OCH₃, —CH₂—SCH₃, —CH₂—SC₂H₅ or —CH₂—CH₂—SCH₃, most preferably —CH₂—CH₂—OCH₃. In another preferred embodiment said composition comprises a paramagnetic metal ion, and said paramagnetic metal ion is preferably a compound comprising Gd³⁺ as a paramagnetic metal ion, preferably a paramagnetic chelate comprising a chelator and Gd³⁺ as a paramagnetic metal ion. In a most preferred embodiment, the composition according to the invention comprises sodium ¹³C₁-L-lactic acid, a trityl radical of formula (1) and a paramagnetic metal ion. Said composition may further comprise a solvent or solvents; preferably an aqueous carrier and most preferably water is used as a solvent. The aforementioned compositions can be used for obtaining hyperpolarised ¹³C₁-lactic acid by dynamic nuclear polarisation (DNP) with a high polarisation level. Said hyperpolarised ¹³C₁-lactic acid can be converted into hyperpolarised ¹³C₁-lactate by dissolution with a base, e.g. NaOH.

Yet another aspect of the invention is a composition comprising hyperpolarised sodium ¹³C₁-lactate or hyperpolarised ¹³C₁-lactic acid, a trityl radical and optionally a paramagnetic metal ion, wherein said composition is obtained by dynamic nuclear polarisation. In a preferred embodiment, said hyperpolarised sodium ¹³C₁-lactate is hyperpolarised sodium ¹³C₁-L-lactate and said hyperpolarised ¹³C₁-lactic acid is hyperpolarised ¹³C₁-L-lactic acid.

Yet another aspect of the invention is hyperpolarised sodium ¹³C₁-L-lactate or hyperpolarised sodium ¹³C₁-D-lactate, preferably hyperpolarised sodium ¹³C₁-L-lactate.

Yet another aspect of the invention is an imaging medium comprising hyperpolarised sodium ¹³C₁-lactate and/or hyperpolarised sodium ¹³C₁-D-lactate, preferably sodium ¹³C₁-L-lactate.

The imaging medium according to the invention may be used as imaging medium in ¹³C-MR detection.

The imaging medium according to the invention may be used as imaging medium for in vitro ¹³C-MR detection, e.g. ¹³C-MR detection of cell cultures, samples, ex vivo tissue or isolated organs derived from the human or non-human animal body. For this purpose, the imaging medium is provided as a composition that is suitable for being added to, for instance, cell cultures, samples like urine, blood or saliva, ex vivo tissues like biopsy tissues or isolated organs. Such an imaging medium preferably comprises in addition to the imaging agent hyperpolarised ¹³C-lactate a solvent which is compatible with and used for in vitro cell or tissue assays, for instance DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such an imaging medium but are not required for such a purpose.

Further, the imaging medium according to the invention may be used as imaging medium for in vivo ¹³C-MR detection, i.e. ¹³C-MR detection carried out on living human or non-human animal beings. For this purpose, the imaging medium needs to be suitable for administration to a living human or non-human animal body. Hence such an imaging medium preferably comprises in addition to the imaging agent, i.e. the MR active agent ¹³C-lactate, an aqueous carrier, preferably a physiologically tolerable and pharmaceutically accepted aqueous carrier like water, a buffer solution or saline. Such an imaging medium may further comprise conventional pharmaceutical or veterinary carriers or excipients, e.g. formulation aids such as stabilizers, osmolality adjusting agents, solubilising agents and the like which are conventional for diagnostic compositions in human or veterinary medicine.

### BRIEF DESCRIPTION OF THE DRAWINGS

FIG. 1 depicts signal intensities of ¹³C₁-lactate, ¹³C₁-alanine, ¹³C₁-pyruvate and ¹³C₁-bicarbonate over time detected from ¹³C-MR spectroscopy imaging of mice (whole body).

FIG. 2 depicts a stacked plot of 30 ¹³C-MR scans showing the signal intensities of ¹³C₁-lactate (183.7 ppm), ¹³C₁-alanine (177.0 ppm), ¹³C₁-pyruvate (171.6 ppm) over time. The signal intensity of ¹³C₁-bicarbonate is outside the displayed ppm-range and thus not shown.

FIG. 3 depicts signal intensities of ¹³C₁-lactate, ¹³C₁-alanine and ¹³C₁-pyruvate over time detected from ¹³C-MR spectroscopy imaging of mouse livers.

FIG. 4 depicts a combined ¹³C-MR spectrum of 20 separate ¹³C-MR scans showing the signal intensities of ¹³C₁-lactate (183.7 ppm), ¹³C₁-alanine (177.0 ppm), ¹³C₁-pyruvate (171.6 ppm) and ¹³C₁-bicarbonate (30.0 ppm).

FIG. 5 depicts signal intensities of ¹³C₁-lactate, ¹³C₁-alanine, ¹³C₁-pyruvate and ¹³C₁-bicarbonate over time detected from ¹³C-MR spectroscopy imaging of mouse hearts.

The invention is illustrated by the following non-limiting examples:

### EXAMPLES

### Example 1a

### Production of Hyperpolarised Sodium ¹³C₁-Lactate by the DNP Method in the Presence of a Gd-Chelate as Paramagnetic Metal Ion and a Trityl Radical as DNP Agent

To a micro test tube was added sodium ¹³C₁-L-lactate solution (78.5 mg, Aldrich, 50% w/w sodium ¹³C₁-lactate). The cap of the tube was punctured with a needle and the solution was frozen in liquid nitrogen. The tube was put in a flask and connected to a freeze-dryer. After drying the tube contained 41 mg dried sodium ¹³C₁-L-lactate (approx 0.36 mmol, sticky substance). A 145 mM aqueous solution of tris(8-carboxy-2,2,6,6-(tetra(hydroxyethyl)benzo-[1,2-4,5′]-bis-(1,3)-dithiole-4-yl)-methyl sodium salt (trityl radical) which had been synthesised according to Example 7 of WO-A1-98/39277 was prepared and 3.5 μl of this solution were added to the dried sodium ¹³C₁-lactate in the tube. Further, a 5 mM aqueous solution of the Gd-chelate of 1,3,5-tris-(N-(DO3A-acetamido)-N-methyl-4-amino-2-methylphenyl)-[1,3,5]tria-zinane-2,4,6-trione (paramagnetic metal ion) which had been synthesised according to Example 4 of WO-A-2007/064226 was prepared and 2.0 μl of this solution was added to the test tube with the sodium ¹³C₁-lactate and the trityl radical. The resulting composition was sonicated and whirl-mixed to dissolve all compounds. The composition was transferred from the tube to a sample cup and the sample cup was inserted into a DNP polariser. The composition was polarised under DNP conditions at 1.2 K in a 3.35 T magnetic field under irradiation with microwave (94 GHz). Polarisation was followed by solid state ¹³C-NMR and the solid state polarisation was determined to be 20%.

### Example 1b

### Production of an Imaging Medium Comprising Hyperpolarised Sodium ¹³C₁-Lactate

After 60 min dynamic nuclear polarisation, the frozen polarised composition obtained was dissolved in 6 ml phosphate buffer (20 mM, pH 7.4, 100 mg/l EDTA). The pH of the final solution containing the dissolved composition was 7.4±0.1. The sodium ¹³C₁-L-lactate concentration in said final solution was 60±2 mM.

Liquid state polarisation was determined by liquid state ¹³C-NMR at 400 MHz to be 18-20%.

### Example 2

### Production of Hyperpolarised Sodium ¹³C₁-L-Lactate by the DNP Method in the Presence of a Gd-Chelate as Paramagnetic Metal Ion and a Trityl Radical as DNP Agent and Production of an Imaging Medium Comprising Hyperpolarised Sodium ¹³C₁-L-Lactate

Example 2 was carried out as Example 1a, however, a water/glycerol mixture (75:25) was used to prepare the trityl and the Gd-chelate solutions. Solid state polarisation was determined to be 17-20%. The frozen polarised composition obtained was dissolved as described in Example 1b. Liquid state polarisation was determined to be 15-20%. The sodium ¹³C₁-L-lactate concentration in the final solution was 30-50 mM.

### Example 3

### Production of Hyperpolarised Sodium ¹³C₁-L-Lactate by the DNP Method in the Presence of a Gd-Chelate as Paramagnetic Metal Ion and a Trityl Radical as DNP Agent and Production of an Imaging Medium Comprising Hyperpolarised Sodium ¹³C₁-L-Lactate

Example 3 was carried out as Example 1a, however, a water/glycerol mixture (50:50) was used to prepare the trityl and the Gd-chelate solutions. Solid state polarisation was determined to be 25%. The frozen polarised composition obtained was dissolved as described in Example 1b. Liquid state polarisation was determined to be 25%. The sodium ¹³C₁-L-lactate concentration in the final solution was 30 mM.

### Example 4

### Production of Hyperpolarised ¹³C₁-L-Lactic Acid by the DNP Method in the Presence of a Gd-Chelate as Paramagnetic Metal Ion and a Trityl Radical as DNP Agent

1.5 mmol sodium ¹³C₁-L-lactate is dissolved in a cooled solution of 500 μl concentrated H₂SO₄ in 2 ml water. The resulting mixture is continuously extracted with diethyl ether, the organic phases are combined, dried over MgSO₄ and filtered. The filtrate is concentrated in vacuo and ¹³C₁-L-lactic acid is obtained.

¹³C₁-L-lactic acid (0.4 mmol) is gently melted and tris(8-carboxy-2,2,6,6-(tetra(methoxyethyl)benzo-[1,2-4,5′]bis-(1,3)dithiole-4-yl)methyl sodium salt which was synthesized as described in Example 1 of WO-A-2006/011810 is added to result in a 10 mM concentration of the trityl radical in said ¹³C₁-L-lactic acid. Further, a 5 mM aqueous solution of the Gd-chelate of 1,3,5-tris-(N-(DO3A-acetamido)-N-methyl-4-amino-2-methylphenyl)-[1,3,5]tria-zinane-2,4,6-trione (paramagnetic metal ion) which had been synthesised according to Example 4 of WO-A-2007/064226 is prepared and 2.0 μl of this solution is added to the test tube with the ¹³C₁-L-lactic acid and the trityl radical. The resulting composition is sonicated and whirl-mixed to dissolve all compounds. The composition is transferred from the tube to a sample cup and the sample cup was inserted into a DNP polariser. The composition was polarised under DNP conditions at 1.2 K in a 3.35 T magnetic field under irradiation with microwave (94 GHz). Polarisation was followed by solid state ¹³C-NMR.

### Example 5a

### Production of Hyperpolarised D-Lactic Acid by the DNP Method in the Presence of a Gd-Chelate as Paramagnetic Metal Ion and a Trityl Radical as DNP Agent

To a micro test tube was added 21.7 mg D-lactic acid (0.24 mmol) together with 4 μl water. A 139 mmol/g aqueous solution of tris(8-carboxy-2,2,6,6-(tetra(hydroxyethyl)benzo-[1,2-4,5′]-bis-(1,3)-dithiole-4-yl)-methyl sodium salt (trityl radical) which had been synthesised according to Example 7 of WO-A1-98/39277 was prepared and 2.9 mg of this solution were added to the micro test tube. Further a 14.6 μmol/g aqueous solution of the Gd-chelate of 1,3,5-tris-(N-(DO3A-acetamido)-N-methyl-4-amino-2-methylphenyl)-[1,3,5]tria-zinane-2,4,6-trione (paramagnetic metal ion) which had been synthesised according to Example 4 of WO-A-2007/064226 was prepared and 1.26 mg of this solution was added to the test tube with the D-lactic acid and the trityl radical. The resulting composition was sonicated and whirl-mixed to dissolve all compounds. The composition was transferred from the tube to a sample cup and the sample cup was inserted into a DNP polariser. The composition was polarised under DNP conditions at 1.2 K in a 3.35 T magnetic field under irradiation with microwave (94 GHz).

### Example 5b

### Production of an Imaging Medium Comprising Hyperpolarised D-Lactate

After an overnight dynamic nuclear polarisation, the frozen polarised composition obtained was dissolved in 6 ml phosphate buffer (40 mM, pH 7.3, osmolality match to 200 mM with NaCl, 100 mg/l EDTA, 1 eq. NaOH). The pH of the final solution containing the dissolved composition was 7.1. The D-lactate concentration in said final solution was 40 mM.

Liquid state polarisation was determined by liquid state ¹³C-NMR at 400 MHz to be 14%. The liquid state relaxation (T₁ at 9.4 T) was determined to 44 s.

### Example 6

### In Vitro ¹³C-MR Spectroscopy Using an Imaging Medium Comprising Hyperpolarised Sodium ¹³C₁-Lactate

An imaging medium was prepared as described in Example 1 and 25 μl of the imaging medium (2.7 mM sodium ¹³C₁-lactate) was mixed into 10 M Hep-G2 cells. A dynamic set of ¹³C-MR spectra was acquired every 5 s with a 15 degree RF pulse. ¹³C₁-pyruvate was clearly building up over time. The average conversion was 0.3% with a peak conversion (0.4%) approximately 20 s into the experiment.

### Example 7

### In Vivo ¹³C-MR Spectroscopy in Mice (Whole Body) Using an Imaging Medium Comprising Hyperpolarised Sodium ¹³C₁-Lactate

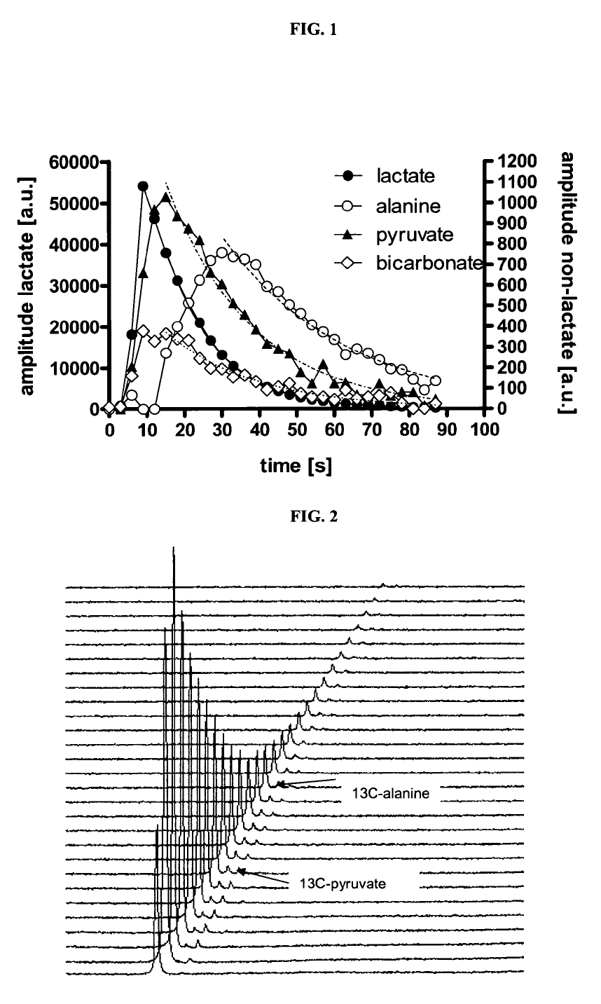

200 μl of an imaging medium which was prepared as described in Example 1 was injected into a C57Bl/6 mouse over a time period of 6 s. The sodium ¹³C₁-lactate concentration in said imaging medium was 60-90 mM and 3 animals were used in the experiment. A rat size whole body coil (tuned for proton and carbon) was placed over the animal and ¹³C-MR spectroscopy was carried out in a 9.4 T magnet. A dynamic set of ¹³C-MR spectra (in total 30) was acquired every 3 s with a 15 degree RF pulse. A significant amount of metabolism was seen with ¹³C₁-pyruvate (approximately 2% of the ¹³C₁-lactate signal) being the earliest peak, followed by ¹³C₁-alanine (approximately 1.5% of the ¹³C₁-lactate signal) at a later point of time. ¹³C₁-bicarbonate (approximately 0.5% of the ¹³C₁-lactate signal) was observable at a similar peak time as ¹³C₁-pyruvate (FIG. 1). FIG. 2 shows a stacked plot of all the 30 acquired spectra. The following decay times were calculated from the MR spectra: ¹³C₁-pyruvate 23 s, ¹³C₁-alanine 33 s and ¹³C₁-bicarbonate 24 s.

### Example 8

### In Vivo ¹³C-MR Spectroscopy in Mice (Liver) Using an Imaging Medium Comprising Hyperpolarised Sodium ¹³C₁-Lactate

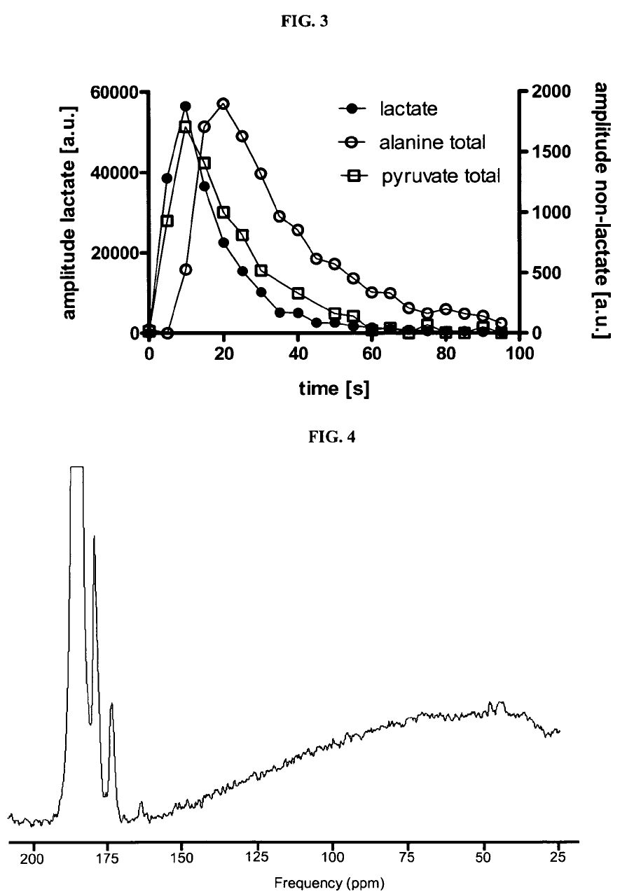

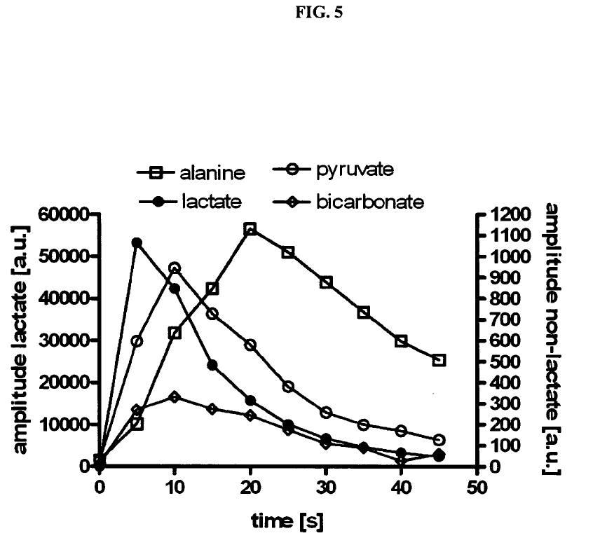

200 μl of an imaging medium which was prepared as described in Example 1 was injected into a C57Bl/6 mouse over a time period of 6 s. The sodium ¹³C₁-lactate concentration in said imaging medium was about 60 mM. A surface coil (tuned for proton and carbon) was positioned over the liver of the animal and ¹³C-MR spectroscopy was carried out in a 9.4 T magnet. A dynamic set of ¹³C-MR spectra (in total 20) was acquired every 5 s with a 30 degree RF pulse. Again a significant amount of metabolism was seen including ¹³C₁-pyruvate (approximately 3% of the ¹³C₁-lactate signal), followed by ¹³C₁-alanine (approximately 3.5% of the ¹³C₁-lactate signal) at a later point of time (FIG. 3). Only very low levels of ¹³C₁-bicarbonate were observed which can be seen in FIG. 4 at 30 ppm. FIG. 4 shows a combined spectrum of the 20 collected MR spectra.

### Example 9

### In Vivo ¹³C-MR Spectroscopy in Mice (Heart) Using an Imaging Medium Comprising Hyperpolarised Sodium ¹³C₁-Lactate

200 μl of an imaging medium which was prepared as described in Example 1 was injected into a C57Bl/6 mouse over a time period of 6 s. The sodium ¹³C₁-lactate concentration in said imaging medium was about 60 mM and 2 animals were used in the experiment. A surface coil (tuned for proton and carbon) was positioned over the heart of the animal and ¹³C-MR spectroscopy was carried out in a 9.4 T magnet. A dynamic set of ¹³C-MR spectra (in total 20) was acquired every 5 s with a 30 degree RF pulse. Again a significant amount of metabolism was seen including ¹³C₁-pyruvate (approximately 2% of the ¹³C₁-lactate signal), followed by ¹³C₁-alanine at a later point of time. ¹³C₁-bicarbonate (approximately 0.5% of the ¹³C₁-lactate signal) was observable at a similar peak time as ¹³C₁-pyruvate (FIG. 5).

What is claimed is:

1. A method of ¹³C-MR detection using an imaging medium comprising hyperpolarised ¹³C-lactate.

2. The method according to claim 1, wherein signals of ¹³C-lactate, ¹³C-pyruvate and ¹³C-alanine, preferably signals of ¹³C-lactate, ¹³C-pyruvate, ¹³C-alanine and ¹³C-bicarbonate are detected.

3. The method according to claim 2, wherein said signals are used to generate a metabolic profile.

4. The method according to claim 3, wherein said method is a method of in vivo ¹³C-MR detection and said metabolic profile is a metabolic profile of a living human or non-human animal being

5. The method according to claim 3, wherein said method is a method of in vitro ¹³C-MR detection and said metabolic profile is one of a metabolic profile of cells in a cell culture, of body samples, of ex vivo tissue, and of an isolated organ.

6. A composition comprising one of sodium ¹³C₁-lactate, ¹³C₁-lactic acid, a trityl radical and, optionally, a paramagnetic metal ion.

7. The composition according to claim 6, wherein said sodium ¹³C₁-lactate or ¹³C₁-lactic acid is sodium ¹³C₁-L-lactate or ¹³C₁-L-lactic acid.

8. The composition according to claim 6, wherein said paramagnetic metal ion is present and is a paramagnetic chelate comprising Gd³⁺.

9. The composition according to claim 6, wherein said trityl radical is a trityl radical of formula (1) 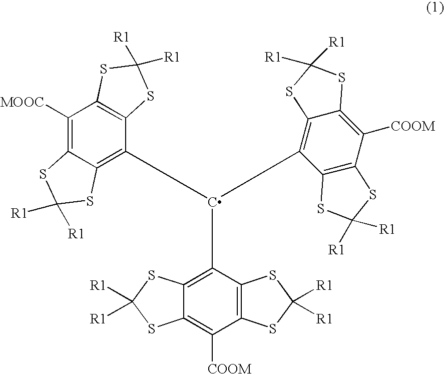
wherein
M represents hydrogen or one equivalent of a cation; and
R1 which is the same or different represents a straight chain or branched C₁-C₆-alkyl group optionally substituted by one or more hydroxyl groups or a group —(CH₂)ₙ—X—R2,
wherein n is 1, 2 or 3;
X is O or S; and
R2 is a straight chain or branched C₁-C₄-alkyl group, optionally substituted by one or more hydroxyl groups.

10. The composition according to claim 6 for use in dynamic nuclear polarisation.

11. A composition comprising one of hyperpolarised sodium ¹³C₁-lactate and hyperpolarised ¹³C₁-lactic acid, a trityl radical and, optionally, a paramagnetic metal ion, wherein said composition is obtained by dynamic nuclear polarisation of the composition of claim 6.

12. An imaging medium comprising hyperpolarised sodium ¹³C₁-lactate, preferably sodium ¹³C₁-L-lactate.

13. The imaging medium according to claim 12 for use in the method of claim 1.

14. Hyperpolarised sodium ¹³C₁-L-lactate.
